(19) United States
(12) Patent Application Publication
(10) Pub. No.: US 2023/0329564 A1
(43) Pub. Date: Oct. 19, 2023

(54) APPARATUS AND METHOD FOR MEASURING BLOOD IN LIQUID

(71) Applicant: Paree Group Oy, KAUHAJOKI AS (FI)

(72) Inventor: Tapio Vaarala, VTT (FI)

(21) Appl. No.: 18/025,981

(22) PCT Filed: Sep. 17, 2021
(86) PCT No.: PCT/FI2021/050616

§ 371 Date: Mar. 13, 2023

(30) Foreign Application Priority Data
Sep. 21, 2020 (FI) 20205913

(51) Int. Cl. A61B 5/02; G01N 33/49; G01N 21/3577

(52) U.S. Cl. CPC A61B 5/02042; G01N 33/49; G01N 21/3577; A61B 2505/05

(57) ABSTRACT

The present solution relates to an apparatus for measuring blood in liquid. The apparatus comprises at least one first light source, at least one first detector for measuring a light parameter in a wavelength range that is between 600 nm and 700 nm, a transparent flow-through channel and a measuring device for measuring the total amount of liquid. The transparent flow-through channel is in the space between the at least one first light source and the at least one first detector. The present solution also relates to a method for measuring blood in liquid.

### FIELD OF THE INVENTION

The present invention relates to an apparatus for measuring blood in liquid.

### BACKGROUND OF THE INVENTION

During a medical or surgical operation it is usually a physician or a surgeon who gives an estimate about the amount of blood in liquid without using any devices, i.e. it is estimated how much a patient is bleeding. Inadequate information about bleeding may impair decision-making about a patient's proper treatment and may lead to a slow recovery. For example, a misjudgement about bleeding in a childbirth may be fatal.

If information about bleeding is inadequate a patient may receive too much blood in a blood transfusion, or too little blood. Further, there may be unnecessary use of blood products, i.e. the blood products are wasted.

### BRIEF DESCRIPTION OF THE INVENTION

An object of the present invention is to provide a method and an apparatus for implementing the method so as to solve the above problems. The objects of the invention are achieved by a method and an apparatus which are characterized by what is stated in the independent claims. The preferred embodiments of the invention are disclosed in the dependent claims.

The invention is based on the idea of metering light that passes through blood-containing liquid in order to find out how much blood the liquid contains.

An advantage of the apparatus and the method of the invention is that the amount of blood can be detected accurately since numerical values are available. Thus, it is possible to know how much blood a patient has lost and keep blood transfusion on a right level. As a consequence, a risk of an incorrect transfusion volume and related complications decrease. Further, precious blood is saved when it is not used in vain.

The apparatus is for measuring blood in liquid. The apparatus is usually used during a medical or surgical operation in order to measure how much a patient has lost blood in the operation. One important use of the apparatus is to meter blood loss during childbirth.

The apparatus comprises at least one first light source, at least one first detector for measuring a light parameter, a transparent flow-through channel and a measuring device for measuring the total amount of liquid. The transparent flow-through channel is located in the space between the light source and the detector.

The apparatus may comprise one first light source but one or more detectors. The first light source may be e.g. a light source emitting white light. One detector may measure the light parameter in a wavelength range that is between 600 nm and 700 nm. Another detector may measure the light parameter in a wavelength range that is between 450 nm and 550 nm.

The apparatus may comprise at least two first light sources emitting light whose wavelength range differ from each other, at least two detectors for measuring a light parameter, a transparent flow-through channel and a measuring device for measuring the total amount of liquid. One of the at least two first light sources emits light whose wavelength range is between 450 nm and 550 nm. One of at least two first light sources emits light whose wavelength range is between 600 nm and 700 nm.

In practice, the number of the first light sources and detectors may vary. There may be, for example, one detector per one light source, one detector per more than one light source, or more than one detector per one light source. One detector per more than one light source or more than one wavelength range may come into question when the detector measures in pulses.

The apparatus may have two sets of the first light sources and corresponding detectors. One set may measure the blood content that is between 0 and 20 volume-% and the other set may measure the blood content that is between 20 and 100 volume-%. As the apparatus measures light that passes through liquid the results must be calibrated in order to convert them into concentrations.

At least two different wavelengths are used since gas, e.g. air bubbles, in liquid is indistinguishable from blood in liquid when only one wavelength range is used. In practice, gas in liquid decreases light intensity. It may happen e.g. that the flow-through channel is filled with gas but the measurement result is the same as with liquid including a small amount of blood.

By using two wavelength ranges it is possible to distinguish blood from liquids other than blood and distinguish gas from blood. Blood is distinguishable from liquids other than blood by using a wavelength range that is between 600 nm and 700 nm. Blood is distinguishable from gas by comparing the strength of the signals received by using a wavelength range from 450 nm to 550 nm and a wavelength from 600 nm to 700 nm. However, if gas is removed from blood before the measurement it is possible to use only one wavelength, i.e. the wavelength from 600 nm to 700 nm. Removing gas from blood means in this context removing separate gas portions, such as air or steam that are capable of forming bubbles or spaces without liquid. Gas that has dissolved in liquid or exists in cells may be present during the measurement.

The wavelength range that is defined to be between 450 nm and 550 nm may be a range that excludes the end points of the range, i.e. the wavelength may be more than 450 nm and less than 550 nm, i.e. 450 nm<λ<550 nm (A denotes the wavelength). The wavelength range that is defined to be between 600 nm and 700 nm may be a range that excludes the end points of the range, i.e. the wavelength may be more than 600 nm and less than 700 nm, i.e. 600 nm<λ<700 nm.

The light sources may comprise LED lights that emit specific wavelengths. The detectors for measuring a light parameter may measure light intensity or light spectrum or both. In other words, the light detectors may measure the intensity of light that has passed through liquid, or the light detectors may measure wavelengths that has passed through liquid/wavelengths that has absorbed in liquid, or the measurement is accomplished in both the above-mentioned ways. The detectors may be photodiodes that convert light into electric currents.

There is a space between the light source/s and the detector/s where a transparent flow-through channel is set, i.e. the transparent flow-through channel is in the space between the light source/s and the detector/s. Liquid, which contains blood, flows through the transparent flow-through channel. The transparent flow-through channel may be a collection container tube, or a tubular element joined to the collection container tube. The tubular element may be a short transparent piece that is joined to the collection container tube.

The measuring device measures the total amount of liquid that has passed through the transparent flow-through channel. The measuring device may be, for example, a weighing appliance or a flow meter. The accuracy of the measurement may be better if the apparatus and the measuring device gives information about the same liquid composition, i.e. it is known when the apparatus has measured the light parameter and the measuring device is timed accordingly.

The apparatus comprises at least two second light sources and at least one second light detector for measuring a quantity that represents oxygen saturation since the amount of oxygen in blood has an effect on the absorption. The quantity should be calibrated in order to find out the blood saturation. The second light sources and the second light detectors may be at the same flow-through channel and assembled in the same manner as the first light sources and the first light detectors. One of the second light sources emits red light whose wavelength may be 660 nm. One of the second light sources may emit infrared light whose wavelength may be 910 nm. The oxygen saturation is a ratio of the absorbed red light to the absorbed infrared light.

A software receives the results of the measurements through an A/D converter from the detectors and the metering device and calculates the amount of blood and the oxygen saturation in the certain liquid batch. However, if the detector/s is/are e.g. a photon counter/photon counters the A/D converter is not required. Also the flow meter can be put into practice by calculating pulses so that the A/D converter is not required.

The apparatus may be connected to a user interface that may act as a display device and/or a device for entering parameters, such as alarm limits. The user interface may be only for the apparatus, or the user interface may serve a whole arrangement that collects liquid from a patient.

Thus, the apparatus may be used in connection with a collection arrangement that draws liquid from a suction site by using vacuum. The collection arrangement may comprise a collection container and a collection container tube. The collection container is connected to a vacuum source. The collection container tube is connected to the collection container. Vacuum that prevails in the collection container draws liquid from the suction site through the collection container tube.

The apparatus may also be used in connection with an arrangement that removes liquid directly to a waste management system.

Further, the apparatus may be used without vacuum when e.g. a pump circulates liquid, or liquid flows freely through a tube that passes through the apparatus for measuring blood in liquid.

### BRIEF DESCRIPTION OF THE DRAWINGS

In the following the invention will be described in greater detail by means of preferred embodiments with reference to the attached drawings, in which

FIG. 1 shows a schematic view of an apparatus;

FIG. 2 shows a first schematic view of an arrangement related to the apparatus;

FIG. 3 shows a second schematic view of an arrangement related to the apparatus;

FIG. 4 shows a third schematic view of an arrangement related to the apparatus;

FIG. 5 shows a fourth schematic view of an arrangement related to the apparatus;

FIG. 6 shows a fifth schematic view of an arrangement related to the apparatus;

FIG. 7 shows a sixth schematic view of an arrangement related to the apparatus;

FIG. 8 shows a seventh schematic view of an arrangement related to the apparatus;

FIG. 9 shows an eighth schematic view of an arrangement related to the apparatus;

FIG. 10 shows a ninth schematic view of an arrangement related to the apparatus;

FIG. 11 shows an apparatus for collecting liquid;

FIG. 12 shows a collection canister and a collection liner;

FIG. 13 shows a manifold;

FIG. 14 shows another collection canister and collection liner.

### DETAILED DESCRIPTION OF THE INVENTION

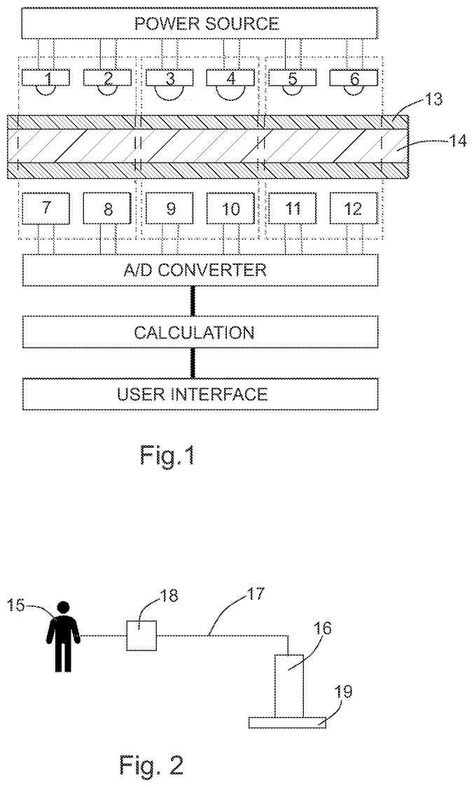

FIG. 1 shows a schematic view of an apparatus. The apparatus comprises light sources 1 to 6 and detectors 7 to 12. The light sources 1 to 6 may be LED lights. The detectors 7 to 12 may be photodiodes. There is a transparent tubular flow-through channel 13 between the light sources 1 to 6 and the detectors 7 to 12. Liquid 14 comprising blood is arranged to flow inside the channel 13.

The light sources 1, 3 emit light having a wavelength range between 450 and 550 nm. The light sources 2, 4 may emit light having a wavelength range between 600 and 700 nm. Blood is distinguishable from liquids other than blood by using a wavelength range that is between 600 nm and 700 nm.

The light source 5 emits red light that may have a wavelength of 660 nm. The light source 6 emits infrared light that may have a wavelength of 910 nm.

The measuring frequency of the detectors may be 10 kHz.

By using the above-mentioned arrangement plain liquid, blood in liquid and gas in liquid, e.g. air, are separable. Blood in liquid is measurable by having a wavelength range between 600 and 700 nm, i.e. by using the light sources 2, 4 and the corresponding detectors 8, 10. Gas in liquid can be detected by comparing the strength of the signal that is measured by using the wavelength range between 450 nm and 550 nm and the wavelength range between 600 nm and 700 nm. In other words, gas in liquid is detected by comparing the strengths of the signals from the detectors 7, 9 to the strength of the signals from the detectors 8,10.

Oxygen in blood has an effect on the absorption of light. Therefore, a quantity that represents oxygen saturation is measured from liquid flowing in the transparent flow-through channel 13 by using the light sources 5, 6 and the corresponding detectors 11, 12.

An A/D converter converts analogue signals of the detectors 7 to 12 into digital signals. The measurement results obtained from the detectors 7 to 12 are used together with the total amount of liquid to calculate the amount of blood in liquid. A user interface shows the calculated results. The user interface may also be utilized for setting alarm limits, for example.

FIG. 1 shows a basic arrangement of the apparatus. In practise, the apparatus may comprise one first light source but one or more detectors. The first light source may be e.g. a light source emitting white light. One detector may measure the light parameter in a wavelength range that is between 600 nm and 700 nm. Another detector may measure the light parameter in a wavelength range that is between 450 nm and 550 nm. However, the detector measuring the wavelength range between 450 nm and 550 nm may not be necessary if gas, such as air bubbles, are removed before the measurement.

FIG. 2 shows one alternative about an arrangement for collecting liquid from a patient 15. The patient 15 may be a human or an animal. The arrangement comprises a collection container 16 that collects liquid through a collection container tube 17 from the patient 15 during a medical or a surgical operation by using vacuum. The arrangement comprises an apparatus 18 for measuring blood in liquid. In order to find out how much liquid is collected the collection container 16 is weighed by a weighing appliance 19.

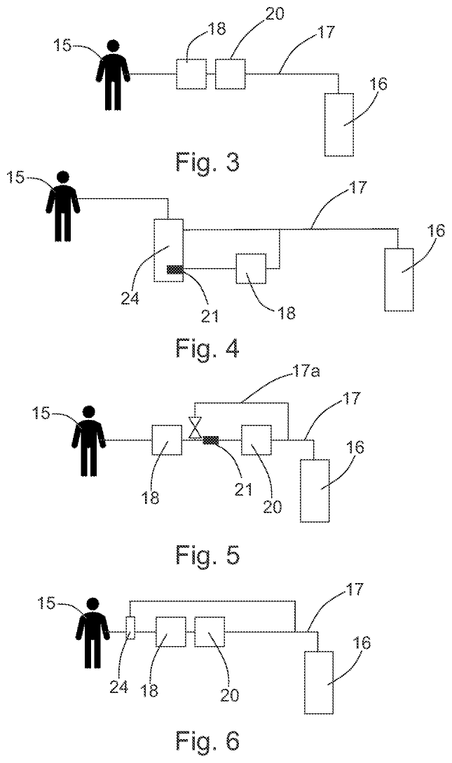

FIG. 3 shows one alternative about an arrangement for collecting liquid from a patient 15. The collection container tube 17 is provided with the apparatus 18 for measuring blood in liquid and a flow meter 20. The flow meter 20 measures the total amount of liquid that has passed it during a certain period.

FIG. 4 shows one alternative about an arrangement for collecting liquid from a patient 15. An intermediate container 24 is provided with a pump 21 which pumps liquid from the intermediate container 24 to the collection container tube 17 that passes through the apparatus 18. Gas and liquid are separated in the intermediate container 24 in such a manner that only liquid runs through the apparatus 18. It is possible to use only one wavelength range, i.e. the range between 600 and 700 nm in this case since gas does not disturb the measurement.

FIG. 5 shows one alternative about an arrangement for collecting liquid from a patient 15. The collection container tube 17 is provided with the apparatus 18, a pump 21 and a flow meter 20, which may be a propeller-type flowmeter. The pump 21 may not be necessary in every case. The collection container tube 17 is provided with a by-pass tube 17a downstream the apparatus 18. The by-pass tube 17a conveys gas. There may be a valve in the intersection of the collection container tube 17 and the by-pass tube 17a. Gas does not disturb the flowmeter 20 in this case.

FIG. 6 shows one alternative about an arrangement for collecting liquid from a patient 15. The collection container tube 17 may be branched in two after an intermediate container 24 in such a manner that one branch removes gas and the other conveys liquid. The latter branch is provided with the apparatus 18 and a flow meter 20. Gas does not disturb the apparatus 18 and the flowmeter 20. It is possible to use only one wavelength range, i.e. the range between 600 and 700 nm in this case since gas does not disturb the measurement.

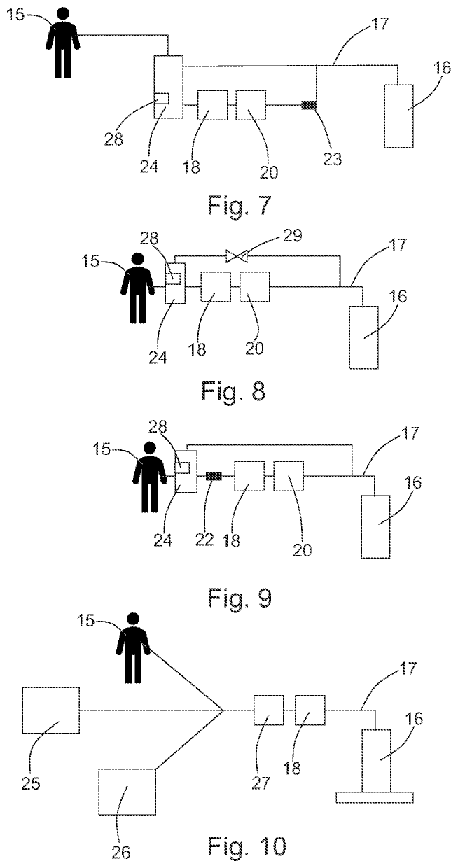

FIG. 7 shows one alternative about an arrangement for collecting liquid from a patient 15. The collection container tube 17 downstream an intermediate container 24 is provided with the apparatus 18, a flow meter 20 and a peristaltic pump 23. The peristaltic pump 23 pumps a fixed amount of liquid per rotation. A liquid level indicator 28 having an upper and lower limit in the intermediate container 24 triggers the pump to start pumping.

FIG. 8 shows one alternative about an arrangement for collecting liquid from a patient 15. There is an intermediate container 24 provided with a liquid level indicator 28 having an upper and lower limit. The collection container tube 17 is branched in two downstream the intermediate container 24. The first tube is provided with a shut-off valve 29 and the second tube is provided with the apparatus 18 and a flow meter 20. The liquid level indicator 28 regulates opening and closing of the shut-off valve 29 so that liquid does not flow through the shut-off valve 29. It is possible to detect only one wavelength range, i.e. the range between 600 and 700 nm in this case since gas does not disturb the measurement.

FIG. 9 shows one alternative about an arrangement for collecting liquid from a patient 15. There is an intermediate container 24 provided with a liquid level indicator 28 having an upper and lower limit. The collection container tube 17 is branched in two. One of the branches is provided with the apparatus 18 and a flow meter 20. A pump 22 pumps liquid and stops if there is no liquid. A flow meter may be integrated in the pump. It is possible to detect only one wavelength range, i.e. the range between 600 and 700 nm in this case since gas does not disturb the measurement.

FIG. 10 shows one alternative about an arrangement for collecting liquid. In addition to collecting liquid from the patient 15 the arrangement may comprise a device 25 for squeezing and a pouch 26. The pouch 26 is for collecting liquid from a surgical drape. As the liquid may contain solid matter, such as bone fractures, there may be a filter 27 before the apparatus 18.

The device 25 for squeezing is arranged to squeeze bloody surgical towels. The squeezing operation may be combined with counting of the towels. The surgical towels may be provided with an electronic identifier so that each of them can be found and squeezed.

Since blood-containing liquid from different sources is led through the apparatus 18 for measuring blood in liquid it is possible to get the total amount of blood that has bled from a patient.

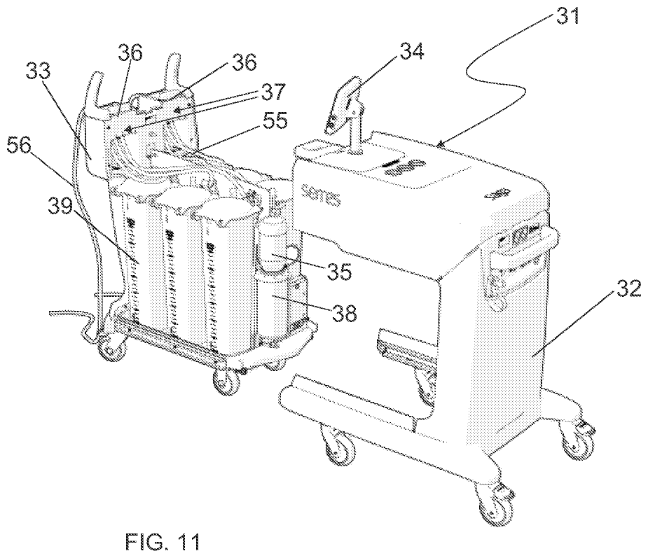

FIG. 11 shows an apparatus 31 for collecting liquid from a patient. The apparatus 31 comprises a control unit 32 and a movable cart 33. The control unit 32 comprises a display unit 34. The display unit 34 may comprise a touch screen acting both for displaying and for entering control parameters.

There are manifolds 36 on the movable cart 33. Valves of the manifolds 36 are operated by suitable actuators, such as motors 37. The movable cart 33 comprises canisters 39 for collecting liquid. There is a weighing appliance under the canisters 39. The movable cart may comprise a cartridge 35 and a reservoir 38 for a solidifying agent.

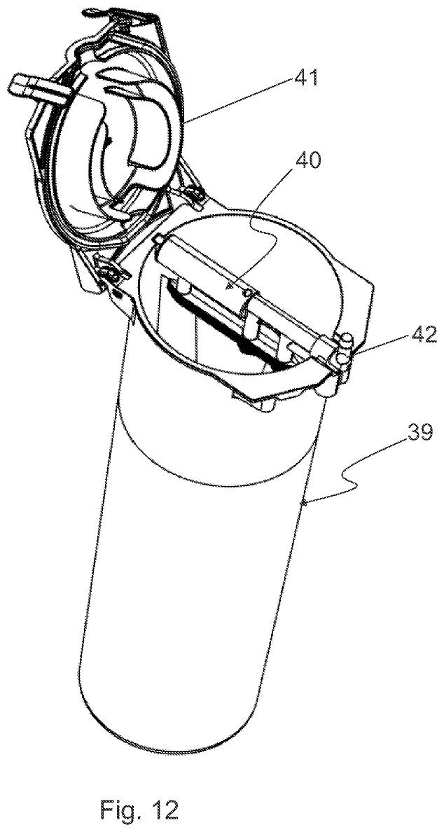

FIG. 12 shows an example of a collection liner and a canister, which are used with the apparatus 31 of FIG. 11, in a perspective view. The collection liner 40 is used with a canister 39 having an openable lid 41. The collection liner 40 is placed inside the canister 39 and an inlet 42 for the collection container tube extends outside the canister 39.

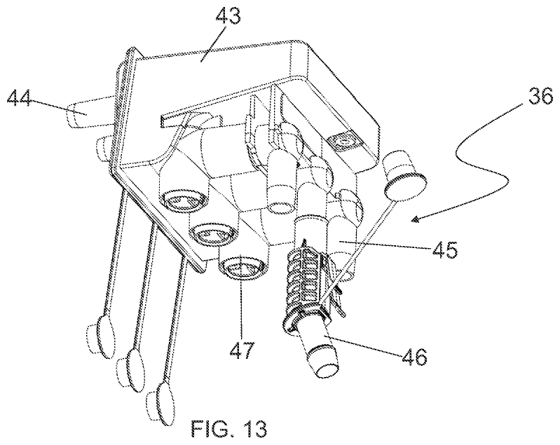

FIG. 13 shows a manifold 36, which may be used with the apparatus 31 of FIG. 11. The manifold 36 comprises a housing 43, patient ports 44 for connecting a patient tube and ports 45 for collection containers tubes. One of the ports 45 is provided with a coupling 46 for a collection container tube in order to illustrate the use of the coupling 46. Each port 45 is provided with a valve 47.

The collection container tubes 55 extend from the ports 45 to the inlet 42. The apparatus for measuring blood in liquid may be in the vicinity of the canister 39 in such a manner that the collection container tube 55 passes through the apparatus 18 for measuring blood in liquid. Each collection container tube 55 may comprise the apparatus 18.

Alternatively, the apparatus may be between the patient and the manifold 36 at the patient tube 56 in such a manner that the collection container tube 56 passes through the apparatus 18 for measuring blood in liquid. The flowmeter 20 may be beside the apparatus 18.

All solutions illustrated in FIGS. 1 to 10 may be applied to the apparatus 31 for collecting liquid shown in FIGS. 11 to 13. The solutions may be applied to the apparatus 31 in connection with the collection container tube 55 or the patient tube 56 when applicable.

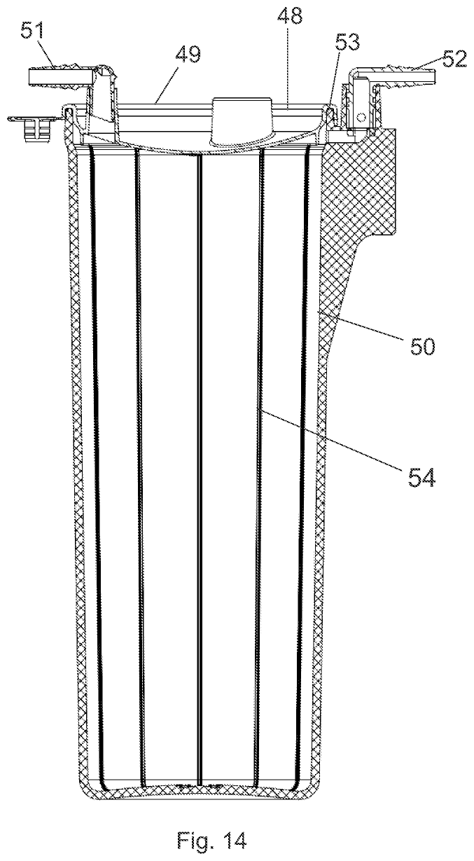

FIG. 14 shows another example of a collection liner and a canister. The collection liner 48 comprises a flexible bag 54, which is fixedly fastened to a lid 49. The flexible bag is placed inside the canister 50 and the rim 53 of the lid 49 closes the canister 50 tightly. The lid 49 comprises an inlet 52 for vacuum and an inlet 51 for a collection container tube, which is at the same time a patient tube, since there is no manifold.

The collection container tube extends from a patient to the inlet 51. The apparatus for measuring blood in liquid may be in the vicinity of the canister 50 in such a manner that the collection container tube passes through the apparatus for measuring blood in liquid. All solutions illustrated in FIGS. 1 to 10 may be applied in connection with the collection liner 48 and the canister 50 shown in FIG. 14.

It will be obvious to a person skilled in the art that, as the technology advances, the inventive concept can be implemented in various ways. The invention and its embodiments are not limited to the examples described above but may vary within the scope of the claims.

1. An apparatus for measuring amount of blood in liquid collected from a patient during a medical or surgical operation, the apparatus is for determining blood loss of the patient during the medical or surgical operation, wherein the apparatus comprises
at least one first light source,
at least one first detector for measuring a light parameter in a wavelength range that is between 600 nm and 700 nm,
a transparent flow-through channel that is in the space between the at least one first light source and the at least one first detector, and
a measuring device for measuring the total amount of liquid.

2. The apparatus according to claim 1, wherein the apparatus comprises another first detector that is arranged to measure a light parameter in a wavelength range that is between 450 nm and 550 nm.

3. The apparatus according to claim 1, wherein the apparatus comprises at least two second light sources and at least a second light detector for measuring oxygen saturation.

4. The apparatus according to claim 3, wherein one of the second light sources emits red light.

5. The apparatus according to claim 3, wherein one of the second light sources emits infrared light.

6. The apparatus according to claim 1, wherein the transparent flow-through channel is a collection container tube, or a tubular element joined to the collection container tube.

7. The apparatus according to claim 1, wherein that the apparatus comprises an A/D converter.

8. The apparatus according to claim 1, wherein the apparatus comprises a software for calculating blood in liquid and oxygen saturation.

9. The apparatus according to claim 1, wherein the apparatus is connected to a user interface.

10. A method for measuring amount of blood in liquid collected from a patient during a medical or surgical operation, the method is for determining blood loss of the patient during the medical or surgical operation, wherein the method comprises
accomplishing a first measurement by detecting light that passes through flowing liquid by using a wavelength range that is between 600 nm and 700 nm, and
metering the total amount of liquid that has flown during the first measurement.

11. The method according to claim 10, wherein the first measurement further comprises detecting light that passes through flowing liquid by using a wavelength range that is between 450 nm and 550 nm.

12. The method according to claim 10, wherein the method comprises removing gas from the liquid before the first measurement.

13. The method according to claim 10, wherein the method comprises accomplishing a second measurement by detecting light that passes through flowing liquid by using at least two different wavelengths.

14. The method according to claim 13, wherein one of the wavelengths belongs to the red light wavelengths and one of the wavelengths belongs to the infrared light wavelengths.

15. The method according to claim 10, wherein the total amount of liquid is metered by weighing or measuring the flow rate.

16. An apparatus for measuring amount of blood in liquid collected from a patient during a medical or surgical operation, the apparatus is for determining blood loss of the patient during the medical or surgical operation, wherein the apparatus comprises
at least one first light source,
at least two first detectors from which detectors one detector is arranged to measure a light parameter in a wavelength range that is between 600 nm and 700 nm and another detector is arranged to measure a light parameter in a wavelength range that is between 450 nm and 550 nm,
a transparent flow-through channel that is in the space between the at least one first light source and the two detectors, and
a measuring device for measuring the total amount of liquid.
